(19) United States
(12) Patent Application Publication
(10) Pub. No.: US 2020/0330252 A1
(43) Pub. Date: Oct. 22, 2020

(54) GASTROINTESTINAL BARRIER IMPLANT AND METHOD OF USE, SURGICAL ANCHOR, AND DELIVERY TOOL FOR SURGICAL ANCHORS

(71) Applicant: Harry FRYDENBERG, Richmond (AU)

(72) Inventor: Harry FRYDENBERG, Richmond (AU)

(73) Assignee: Harry Frydenberg, Richmond (AU)

(21) Appl. No.: 16/304,984

(22) PCT Filed: May 29, 2017
(86) PCT No.: PCT/AU2017/050505

§ 371 Date: Nov. 27, 2018

(30) Foreign Application Priority Data
May 27, 2016 (AU) 2016902009
May 27, 2016 (AU) 2016902010
May 27, 2016 (AU) 2016902011
May 31, 2016 (AU) 2016902071
May 31, 2016 (AU) 2016902072
May 31, 2016 (AU) 2016902073

(51) Int. Cl. A61F 5/00

(52) U.S. Cl. CPC A61F 5/0079; A61F 5/005; A61F 5/0089

(57) ABSTRACT

A gastrointestinal barrier implant including a support, a distal barrier tube extending from the support in a first direction, and a proximal retainer extending from the support in a direction opposite to said first direction, wherein the proximal retainer is adapted to be anchored to an internal wall of the stomach of a user and to extend from the stomach through the pyloric orifice such that the support is located below the pyloric orifice, the support depending from the proximal retainer, and wherein the distal barrier tube depends from the support so as to provide a liner through the duodenum. A surgical anchor suitable for fastening an implant to body tissues in vivo, the anchor comprising a mounting head and a pair of legs extending from the mounting head, distal end portions of the pair of legs remote from the mounting head being adapted to pierce the implant and body tissues, the mounting head being engageable by a coupling device for deploying and retrieving the anchor in use, the pair of legs being movable relative to the mounting head between a piercing configuration assumed by the legs at a first temperature in which the distal ends of the legs are mutually positioned to pierce the implant and tissues for installation of the anchor, and an anchoring configuration assumed by the legs at a higher temperature in which the distal ends of the legs are displaced away from the piercing configuration for securing the implant and tissues in use. A tool for delivering and placing a surgical anchor, comprising a handle carrying a flexible filament of a length to enable its endoscopic introduction to the zone in which the anchor is to be placed, the filament comprising a sheath and a flexible core displaceable longitudinally within the sheath, means carried by the handle and actuable for displacing the core relative to the sheath, the core having at a distal end a coupling device for releasably engaging a head of the surgical anchor, and the sheath having at a distal end a housing for retaining the coupling device and anchor engaged therewith in a retracted condition of the coupling device within the housing, displacement of the core forwardly relative to the sheath causing the coupling device and anchor to move forwardly from the housing for placement of the anchor and release of the anchor from the coupling device.

This application claims priority from Australian Patent Application Nos. 2016902009, 2016902010 and 2016902011 all filed on 27 May 2016 and Australian Patent Application Nos. 2016902071, 2016902072 and 2016902073 all filed on 31 May 2016, the entire contents of which, as originally filed, are incorporated herein by reference.

### FIELD OF THE INVENTION

The invention relates to a gastrointestinal barrier implant and, more specifically, but not exclusively, to a gastrointestinal barrier implant including a support stent which is free of barbs. The invention also relates to a method of implanting a barrier implant. Another aspect of the invention relates to surgical anchors and more particularly to a surgical anchor suitable for being applied endoscopically. Although the invention will be described with reference to the fastening of a gastrointestinal barrier, it is to be understood that the invention in its broader aspects is not limited to that usage. Yet another aspect of the invention relates to a delivery tool for surgical anchors, more particularly to anchors suitable for being applied endoscopically. Although the invention will be described with reference to the delivery and anchors for fastening a gastrointestinal barrier, it is to be understood that the invention in its broader aspects is not limited to that usage.

### BACKGROUND OF THE INVENTION

Barrier Implant

It has been previously proposed to perform bariatric surgery so as to achieve weight loss and/or for the treatment of type-2 diabetes. In particular, it has been proposed to treat obesity by reducing the size of the stomach with a gastric band or through removal of a portion of the stomach by sleeve gastrectomy or biliopancreatic diversion with a duodenal switch. Weight loss surgery may also be performed by resecting and re-routing the small intestine to a small stomach pouch.

U.S. Pat. No. 7,267,694 relates to a bariatric sleeve for limiting absorption of food products in specific parts of the digestive system. More specifically, this patent discloses a gastrointestinal implant device including an anchor having barbs for anchoring the implant device to the muscular pylorus in the stomach. Although this type of bariatric sleeve may assist in addressing some of the drawbacks of more traditional invasive procedures, the applicant has determined there are still disadvantages associated with the bariatric sleeve in that patients may be prone to bleeding and discomfort.

Examples of the present invention seek to provide a gastrointestinal barrier implant which overcomes or at least alleviates one or more disadvantages associated with existing bariatric sleeves.

Surgical Anchor

Conventional surgical staples allow a surgeon to hold together body or bone tissues during surgery. Staples may be applied individually or in series, with stapling legs capable of being bent and thus secured to the tissues using force applied through a stapler. Such surgical staples are designed to be used with open surgery and involve the use of stapling and extracting devices which require unhindered access to a wound.

The aforementioned staples are not suitable for surgical procedures inside the body of a patient that do not involve open or laparoscopic surgery. For example, minimally-invasive or non-invasive surgical procedures typically do not create open wounds of a sufficient size for a surgeon to manoeuvre and position a head of the stapling device inside the body of a patient to apply conventional staples and bending the stapling legs using force applied through the device.

The applicant has determined that it would be advantageous to provide a surgical anchor that is suitable for use endoscopically and for holding together implant and body tissues in vivo. Examples of the present invention seek to at least provide a useful alterative to conventional surgical staples.

Delivery Tool

Conventional surgical staples allow a surgeon to hold together body or bone tissues during surgery. Staples may be applied individually or in series, with stapling legs capable of being bent and thus secured to the tissues using force applied through a stapler. Such surgical staples are designed to be used with open surgery and involve the use of stapling and extracting devices which require unhindered access to a wound.

The aforementioned staples are not suitable for surgical procedures inside the body of a patient that do not involve open surgery. For example, minimally-invasive or non-invasive surgical procedures typically do not create open wounds of a sufficient size for a surgeon to manoeuvre and position a head of the stapling device inside the body of a patient to apply conventional staples and bending the stapling legs using force applied through the device.

The applicant has determined that it would be advantageous to provide a surgical anchor that is suitable for use endoscopically and for holding together implant and body tissues in vivo. Embodiments of the invention provide a tool for delivering such an anchor.

### SUMMARY OF THE INVENTION

Barrier Implant

In accordance with one aspect of the present invention, there is provided a gastrointestinal barrier implant including a support, a distal barrier tube extending from the support in a first direction, and a proximal retainer extending from the support in a direction opposite to said first direction, wherein the proximal retainer is adapted to be anchored to an internal wall of the stomach of a user and to extend from the stomach through the pyloric orifice such that the support is located below the pyloric orifice, the support depending from the proximal retainer, and wherein the distal barrier tube depends from the support so as to provide a liner through the duodenum.

Preferably, the support is in the form of a resilient structure.

In a preferred form, the support is movable between a contracted condition and an expanded condition.

Preferably, the support is arranged to support open the barrier implant to facilitate passage of food through the implant, when the support is in said expanded condition.

Preferably, the support is in the form of a stent.

It is preferred that the support assists in locating the barrier implant by pushing outwardly against the duodenum.

Preferably, the support is arranged to prevent piercing of the wall of the duodenum. More preferably, the support is without barbs.

In a preferred form, the proximal retainer terminates in a collar which is arranged to receive one of more anchors for anchoring the barrier implant to the stomach.

Preferably, when in use, the proximal retainer is anchored to an internal wall of the stomach of the user and extends from the stomach through the pyloric orifice to the support which is located below the pyloric orifice, the support depending from the proximal retainer, and the distal barrier tube depends from the support providing a liner through the duodenum.

Preferably, the implant is packaged into a condensed insert which is inserted into a cavity of a delivery catheter for delivery endoscopically into the user, wherein the cavity retains the support in the contracted condition. More preferably, the delivery catheter is arranged to deliver the implant to the location, and includes a control to remotely eject the insert from the cavity.

In accordance with another aspect of the present invention, there is provided a gastrointestinal barrier implant including a distal barrier tube and a proximal retainer wherein, when in use, the proximal retainer is anchored to an internal wall of the stomach of a user and extends from the stomach through the pyloric orifice, and the distal barrier tube depends from the proximal retainer so as to be supported in place by the proximal retainer whereby the distal barrier tube provides a liner through the duodenum.

In accordance with another aspect of the present invention, there is provided a method of implanting a barrier implant including the steps of:

inserting a delivery catheter into a user;

operating the delivery catheter to remotely eject the barrier implant from the delivery catheter, with the support expanding into position below the pyloric orifice;

drawing the proximal retainer upwardly from the support, through the orifice and into the stomach; and

inserting anchors through the proximal retainer and into the stomach wall.

Preferably, each anchor is adapted to move between an engaging condition and a releasing condition in response to temperature. More preferably, each anchor is formed from a shape memory alloy. In one example, each anchor is formed from nitinol material.

Surgical Anchor

According to an aspect of the present invention, there is provided a surgical anchor suitable for fastening an implant to body tissues in vivo, the anchor comprising a mounting head and a pair of legs extending from the mounting head, distal end portions of the pair of legs remote from the mounting head being adapted to pierce the implant and body tissues, the mounting head being engageable by a coupling device for deploying and retrieving the anchor in use, the pair of legs being movable relative to the mounting head between a piercing configuration assumed by the legs at a first temperature in which the distal ends of the legs are mutually positioned to pierce the implant and tissues for installation of the anchor, and an anchoring configuration assumed by the legs at a higher temperature in which the distal ends of the legs are displaced away from the piercing configuration for securing the implant and tissues in use.

Preferably, at least one leg is curved along its longitudinal length. Preferably, the leg curves away from the opposing leg. Preferably, at least one leg is configured such that a first portion of its length proximate the head curves in a first direction, and a second portion of its length proximate the distal end portion curves in a second direction.

Preferably, in the piercing configuration, the first portion of the at least one leg abuts the opposing leg such that the distal end portions of the pair of legs are not in contact with one another.

Preferably, at least distal end portions of the legs are substantially parallel to one another in the piercing configuration.

Preferably, in the piercing configuration, the distance between the distal end portions of the opposing legs is no more than about 2 mm. Preferably, in the anchoring configuration, the distance between the distal end portions of the opposing legs is at least about 10 mm.

Preferably, the first temperature is substantially of refrigeration temperature. Preferably, the higher temperature is substantially of body temperature.

Preferably, the legs are made from a nickel-titanium alloy.

Preferably, the head is integrally formed with the legs. Preferably, the head is in the form of an eye.

Delivery Tool

In accordance with one aspect of the present invention, there is provided a tool for delivering and placing a surgical anchor, comprising a handle carrying a flexible filament of a length to enable its endoscopic introduction to the zone in which the anchor is to be placed, the filament comprising a sheath and a flexible core displaceable longitudinally within the sheath, means carried by the handle and actuable for displacing the core relative to the sheath, the core having at a distal end a coupling device for releasably engaging a head of the surgical anchor, and the sheath having at a distal end a housing for retaining the coupling device and anchor engaged therewith in a retracted condition of the coupling device within the housing, displacement of the core forwardly relative to the sheath causing the coupling device and anchor to move forwardly from the housing for placement of the anchor and release of the anchor from the coupling device.

Preferably the coupling device is of hook-like form configured to engage an eye in the head of the anchor.

Preferably displacement of the flexible core within the sheath is effected by a slider mounted to the handle and engaged with a piston mounted to the inner end of the core and slidable within the interior of the handle.

In accordance with another aspect of the present invention, there is provided a method of delivering and placing a surgical anchor comprising use of a tool as defined above, wherein an anchor is secured to the coupling device of the tool, the core is displaced longitudinally within the sheath to withdraw the coupling device and anchor into the housing, the flexible filament is inserted via a gastroscope into the patient and manipulated to a delivery site, the core is displaced longitudinally to cause the anchor to partially exit the housing for placement of the anchor into the tissue site, and the core is displaced further to enable the coupling device to be removed from the anchor.

### BRIEF DESCRIPTION OF THE DRAWINGS

The invention is described, by way of non-limiting example only, with reference to the accompanying drawings, in which:

FIG. 1 is a cutaway depiction of a previous gastrointestinal bather implant shown in situ;

FIG. 2 is a cutaway depiction of an example gastrointestinal barrier implant of one aspect of the present invention, shown in situ, which can be fastened using surgical anchors in accordance with another aspect of the present invention;

FIG. 3 shows a side view of a gastrointestinal barrier implant in accordance with an example of the present invention;

FIG. 4 is a detailed side view of an upper portion of the gastrointestinal barrier implant of FIG. 3;

FIG. 5 is a detailed side view of a support of the gastrointestinal barrier implant of FIG. 3;

FIG. 6 is a top perspective view of the gastrointestinal barrier implant of FIG. 3;

FIG. 7 is a detailed side view of a distal end portion of the gastrointestinal barrier implant of FIG. 3;

FIG. 8 is a side view of an upper portion of the gastrointestinal barrier implant of FIG. 3, shown alongside a distal end of a delivery catheter;

FIG. 9 is a side view of a proximal control portion of the delivery catheter;

FIG. 10 shows a perspective top view of a surgical anchor, in accordance with an example of the invention, suitable for fastening the gastrointestinal barrier implant;

FIG. 11 shows a top plan view of the anchor of FIG. 10;

FIG. 12 shows a perspective top view of an alternative example of the surgical anchor;

FIG. 13 shows a top plan view of the anchor of FIG. 12;

FIG. 14 shows a delivery tool, in accordance with another aspect of the present invention, with a coupling device in an extended position;

FIG. 15 shows the tool with the coupling device in an unextended position;

FIG. 16 shows in greater detail a housing for the coupling device, the coupling device being in its extended position;

FIG. 17 is a view similar to FIG. 16 but with the coupling device in its unextended position in the housing;

FIG. 18A shows a cross-sectional representation of the housing and coupling device retaining an anchor within the housing in the unextended position of the coupling device;

FIG. 18B is a view similar to FIG. 18A showing the anchor partly removed from the housing by displacement of the coupling device.

FIG. 18C is a view similar to FIG. 18A showing the anchor fully removed from the housing in the extended position of the coupling device; and

FIG. 19 shows an alternative embodiment of the tool in which the delivery action is spring-loaded.

### DETAILED DESCRIPTION

The applicant has identified that a bariatric sleeve of the kind disclosed in U.S. Pat. No. 7,267,694 may be beneficial for the treatment of obesity in comparison to prior invasive bariatric surgery, but that there are still problems with these bariatric sleeves which may be addressed by the present invention. The entire disclosure of U.S. Pat. No. 7,267,694 is incorporated herein by reference.

In particular, the applicant has identified that previously proposed bariatric sleeves rely on barbs to hold the sleeve in place by piercing the tissue, for example by way of the barbs 210 disclosed in FIG. 2 of U.S. Pat. No. 7,267,694. The applicant has identified that piercing the tissue in this way may lead to tearing of the tissue, bleeding, and ongoing discomfort to the point where the bariatric sleeve must be removed to alleviate these adverse reactions. The applicant has identified that these adverse reactions are due in part to the tissue being insufficiently strong in the area engaged by the barbs (see Figure B of U.S. Pat. No. 7,267,694) and also in part to the barbs failing to provide adequately secure penetration in the tissue so as to remain stationary without undue movement. Advantageously, examples of the invention may address these shortcomings by providing the barrier implant with a proximal retainer which is adapted to be anchored to an internal wall of the stomach above the pyloric orifice where stronger tissue is available for anchorage.

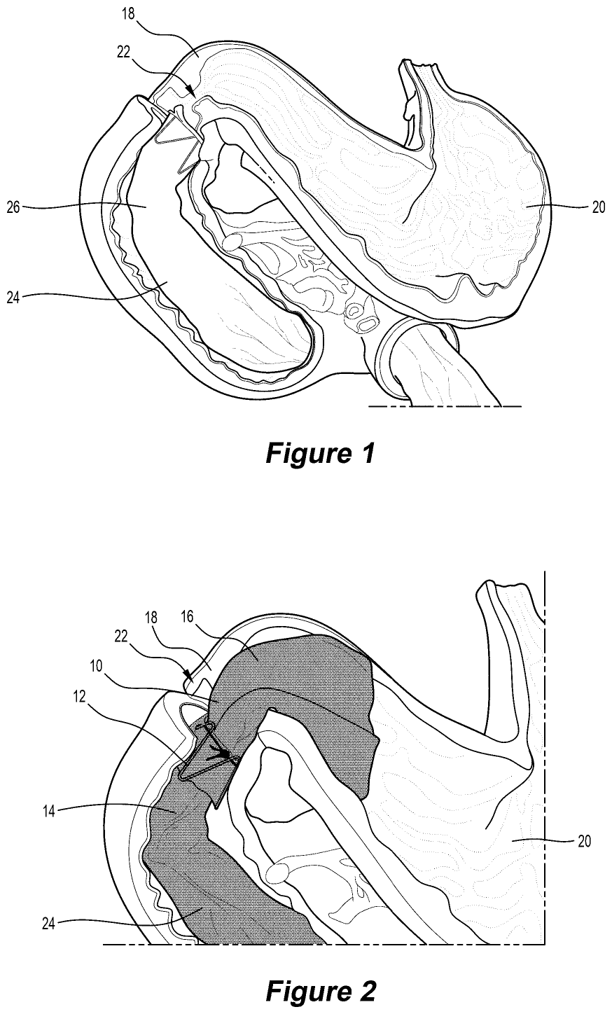

With reference to FIGS. 1 to 9 of the drawings, there is shown a gastrointestinal barrier implant 10 including a support 12, a distal barrier tube 14 extending from the support 12 in a first direction (in the general direction of movement through the gastrointestinal system), and a proximal retainer 16 extending from the support 12 in a direction opposite to the first direction. The proximal retainer 16 is adapted to be anchored to an internal wall 18 of the stomach 20 of a user and to extend from the stomach 20 through the pyloric orifice 22 such that the support 12 is located below the pyloric orifice 22, the support 12 depending from the proximal retainer 16, and the distal barrier tube 14 depending from the support 12 so as to provide a liner through the duodenum 24.

The arrangement of an example gastrointestinal barrier implant 10 of the present invention is demonstrated in FIG. 2 of the drawings, in contrast to an existing bariatric sleeve 26 shown in FIG. 1. In particular, as can be seen, the bariatric sleeve 26 shown in FIG. 1 is anchored in place merely by way of a support 12 located beneath the pyloric orifice 22, whereas the gastrointestinal barrier implant 10 shown in FIG. 2 includes a proximal retainer 16 which extends upwardly from the support 12 so that the proximal retainer 16 can be anchored to the internal wall 18 of the stomach 20, for example, by way of staples or other anchors. In this way, the gastrointestinal barrier implant 10 of the invention is able to be more effectively anchored in place within the patient body without causing the same side effects of the existing bariatric sleeve 26, mainly by way of the gastrointestinal barrier implant 10 being anchored to a stronger anchorage point being in the form of the internal wall 18 above the pyloric orifice 22.

The support 12 may be in the form of a resilient structure which is movable between a contracted condition and an expanded condition. As can be seen in FIG. 2, the support 12 is arranged to support open the barrier implant 10 to facilitate passage of food through the implant 10, when the support 12 is in the expanded condition. The support 12 may be in the form of a stent which assists in locating the barrier implant 10 by pushing outwardly against the duodenum 24. Significantly the support 12 is arranged to prevent piercing of the wall of the duodenum 24, specifically because the support 12 is formed without barbs which have been found by the applicant to be harmful to the patient in existing bariatric sleeves. As the support 12 is movable between the contracted condition and the expanded condition, insertion and removal of the gastrointestinal barrier implant 10 is facilitated by moving the support to the contracted condition during insertion and removal, whereas the support 12 is allowed to self-expand into the expanded condition during ordinary use as shown in FIG. 2 so as to hold open the gastrointestinal barrier implant 10 to facilitate the passage of food through the implant 10.

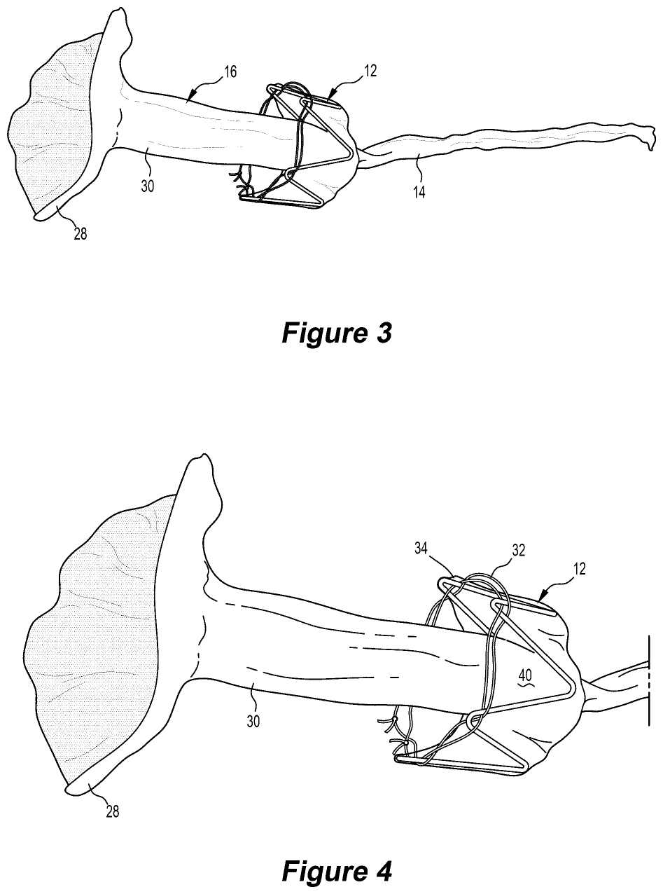

With reference to FIGS. 3 and 4 of the drawings, there is shown a side view of the gastrointestinal barrier implant 10 depicted in the expanded condition but not in situ so as to provide a clear view of the device for describing its structure. The proximal retainer 16 terminates in a collar 28 which is arranged to receive one or more anchors for anchoring the barrier implant 10 to the stomach 20. Specifically, the proximal retainer 16 is in the form of a tube which is flanged outwardly into a generally circular collar 28 such that the collar 28 is able to maintain contact with the internal wall 18 of the stomach 20 so as to act as a funnel for funnelling matter from the stomach into the tube 30 of the proximal retainer 16 so as to then pass into the distal barrier tube 14 to effectively bypass at least part of the duodenum 24. The length of the distal barrier tube 14 may be altered on a case-by-case basis depending on the BMI of the patient and on the specific needs of the patient as would be determined by the surgeon.

As such, when in use, the proximal retainer 16 is anchored to the internal wall 18 of the stomach 20 of the user and extends from the stomach 20 through the pyloric orifice 22 to the support 12 which is located below the pyloric orifice 22 (see FIG. 2), the support 12 depending from the proximal retainer 16, and the distal barrier tube 14 depends from the support 12, thus providing a liner through the duodenum 24.

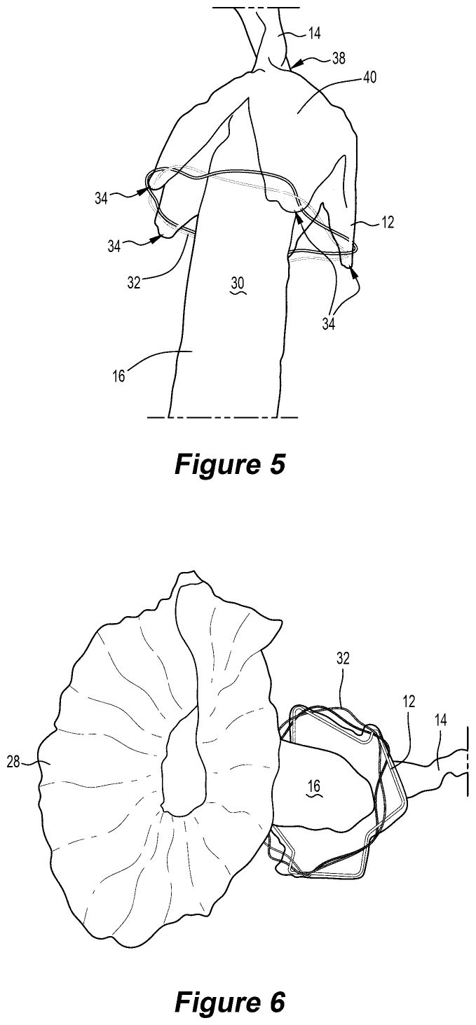

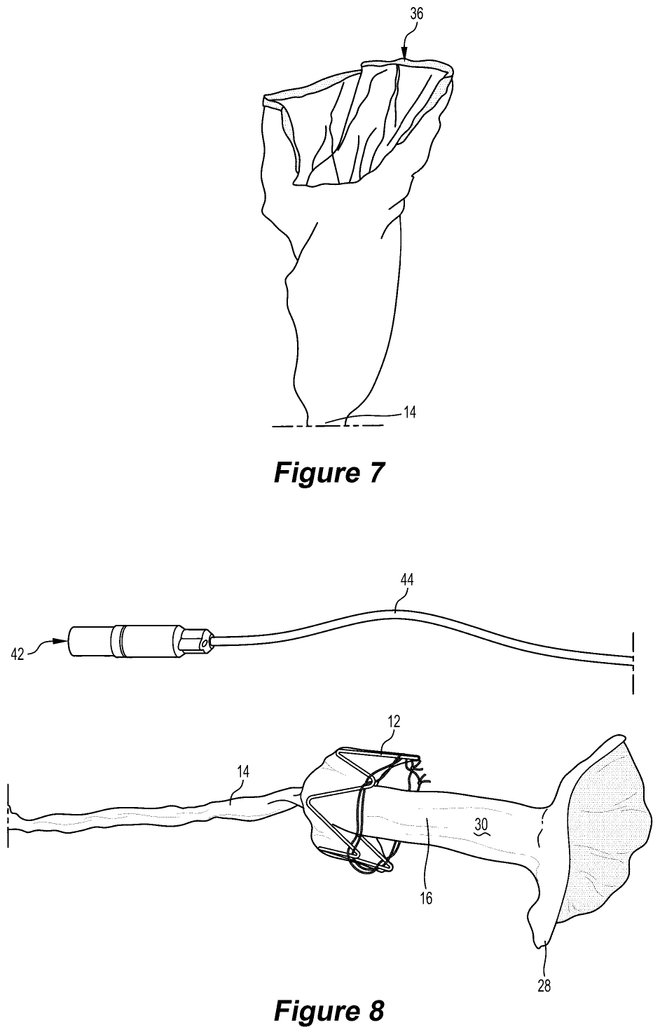

With reference to FIGS. 5 and 6, these drawings show detail of the support 12 which is in the form of a resilient stent which may be formed of wire so as to be biased toward the expanded condition shown in FIG. 5. The wire is bent in an oscillating zig-zag configuration having a series of straight sections of equal length, with an included angle of approximately 30 degrees to 50 degrees between adjacent straight sections. In the example shown, the support 12 is configured to have five upper points and five lower points defined by the bent portions of the wire. A string 32 is threaded between the five upper points 34 so as to limit expansion of the support 12. The tube 30 of the proximal retainer 16 and the distal barrier tube 14 may be continuous and may have the same width where they meet, with the distal barrier tube 14 tapering inwardly toward the distal end 36 shown in FIG. 7. The support 12 is connected to the tube 30 and the distal barrier tube 14 by a cuff which terminates at an upper end along the length of the wire of the support 12 (as seen in FIG. 5) and tapers inwardly and downwardly to an intersection 38 in the form of a generally circular band where the cuff 40 is coupled to the proximal retainer 16 and the distal barrier tube 14. Although not shown in the drawings, the cuff may be provided with one or more apertures surrounding the intersection 38 so as to act as drainage points to prevent accumulation of matter at the intersection 38.

The resilience of the support 12 enables the implant to be packaged into a condensed insert which is inserted into a cavity 42 of a delivery catheter 44 for delivery endoscopically into the user, with the cavity 42 retaining the support 12 in the contracted condition. The delivery catheter 44 may be arranged to deliver the implant 10 to the location, and may include a control 46 to remotely eject the insert from the cavity 42.

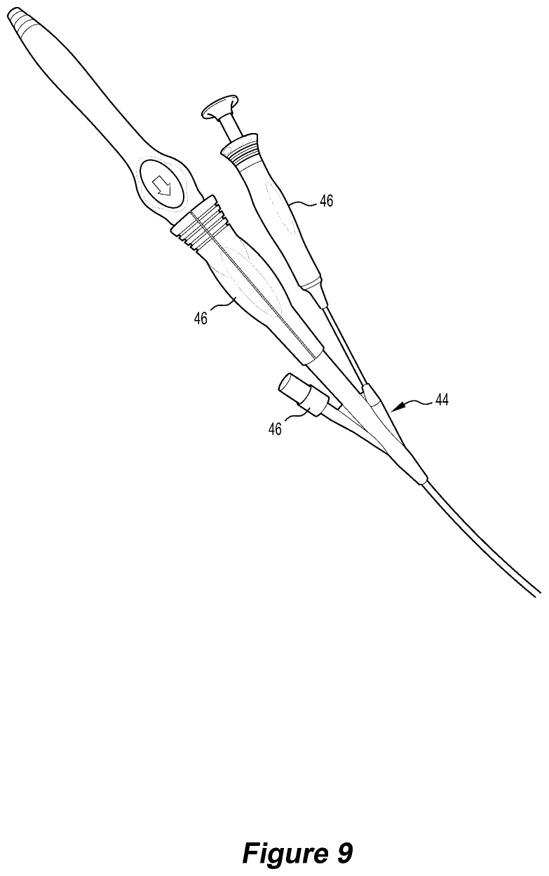

The delivery catheter 44 is in the form of an elongated device having the handheld control 46 at one end and the cavity 42 at the other end, with an elongated tube between the control 46 and the cavity 42. The cavity 42 is shown in FIG. 8 and the handheld control 46 is shown in FIG. 9. The tube between the cavity 42 and the control 46 is sufficiently long such that the tube is able to be inserted into the body of the patient, with the cavity 42 being manipulated into the stomach of the user with control 46 simultaneously being operated by the surgeon outside of the patient so as to eject the insert in place. The control 46 may have a series of different control mechanisms—for example, three control branches are shown in the control 46 depicted in FIG. 9—so as to manipulate the cavity 42 into the correct position inside the stomach of the patient and to thereafter eject the insert. The tube may be in the form of a sheath with cables and/or flexible rods passing through the sheath so as to transmit control signals from the control 46 to the cavity 42. The cavity may have an internal piston which is operated by the control 46 so as to push out the insert, releasing the support 12 from its confinement in the cavity 42 so that the support 12 springs outwardly into place below the pyloric orifice 22 into the expanded condition. The control 46 may also have a specific provision so as to assist in drawing upwardly the proximal retainer 16 away from the support 12 so that the collar 28 of the proximal retainer 16 can be anchored to the internal wall 18 of the stomach 20.

The proximal retainer 16 may be anchored to the internal wall 18 of the stomach by remotely passing one or more anchors through the collar 28. Each of the anchors may be formed from a shape memory alloy, for example from nitinol material. Where a shape memory alloy is used, the anchor may be adapted so as to move into the engaging condition in response to the heat from the body once the anchor penetrates the body tissue, with legs of the anchor expanding outwardly. Also, the anchor may be adapted so as to move into the releasing condition in response to the application of a relatively cold temperature. For example, this may be achieved by applying cooling to an exposed inner end of the anchor such that the engaged arms of the anchor move together to facilitate removal (pulling out) of the anchor. Each of the anchors may be inserted by a specialised fastening tool in conjunction with an endoscope so as to ensure the anchors are inserted into relatively strong and thick tissue, for example the tissue above the pyloric orifice 22. As the tissue is relatively strong, the applicant has determined that it is less likely that tearing of the tissue and extended bleeding would occur, thus alleviating drawbacks associated with existing bariatric sleeves.

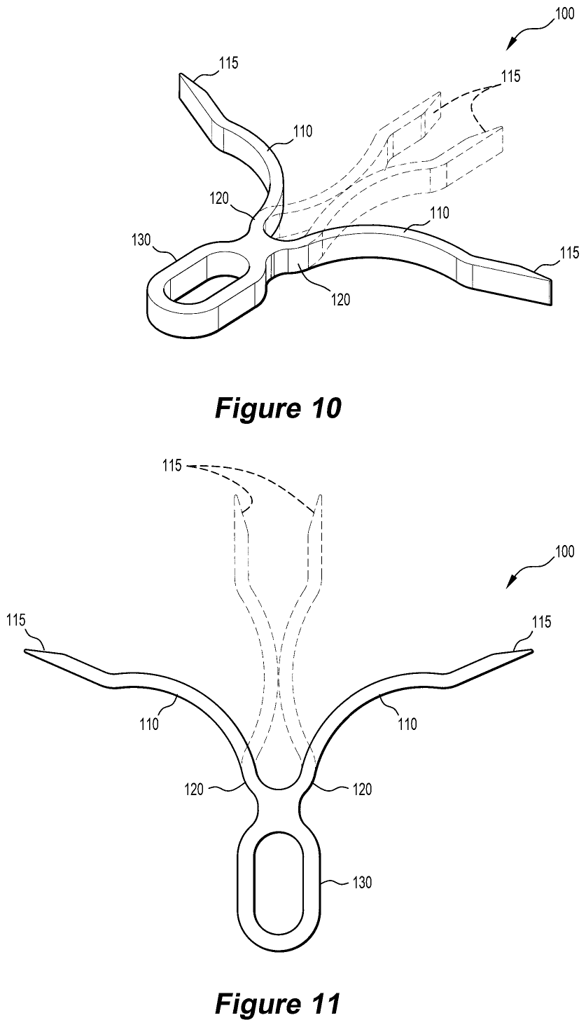

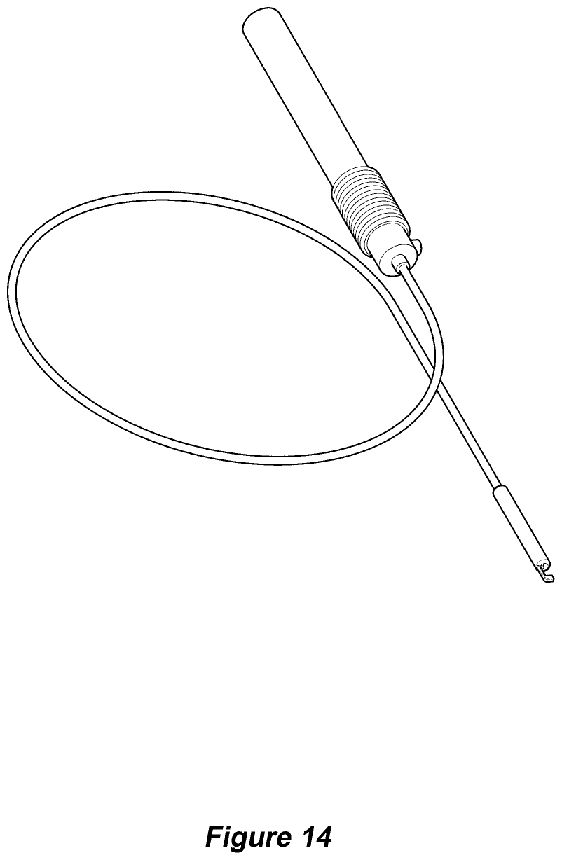

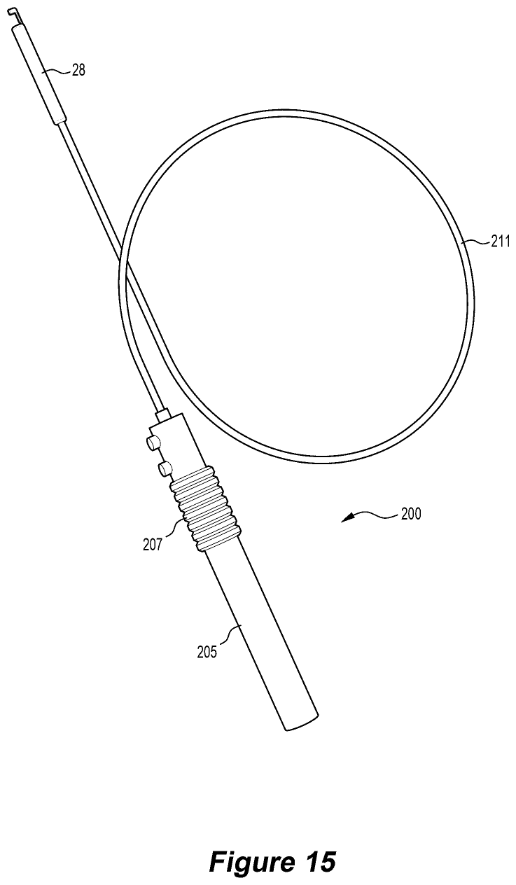

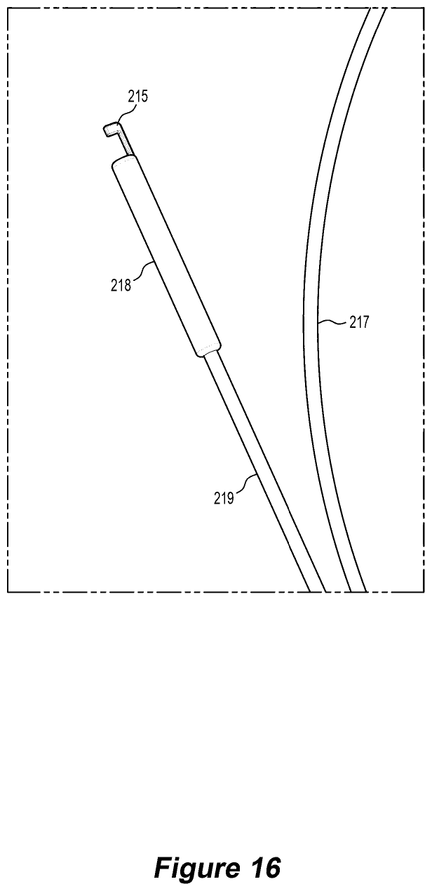

A preferred form of anchor and a preferred form of tool for inserting the anchor will be described with relation to FIGS. 10 to 13, and 14 to 19 below

Once the anchors have been removed, for example after the desired implant period which may be in the order of twelve months, the implant 10 may then be removed from the body by a specialised tool which contracts the support 12 into the contracted condition and then draws the implant 10 upwardly and outwardly through the pyloric orifice 22 and out from the patient's body.

In an alternative example, it has been foreseen that, owing to the implant 10 being anchored in the patient's body by way of the proximal retainer 16 that the feature of the support 12 could possibly be obviated. In such a case, there would be provided the implant 10 including a distal barrier tube 14 and a proximal retainer 16 wherein, when in use, the proximal retainer 15 is anchored to an internal wall 18 of the stomach 20 of the user and extends from the stomach 20 through the pyloric orifice 22, with the distal barrier tube 14 depending from the proximal retainer 16 so as to be supported in place by the proximal retainer 16 whereby the distal barrier tube 14 provides a liner through the duodenum 24.

With reference to FIG. 10, the surgical anchor 100 has a mounting head 130 and a pair of legs 110 extending from the mounting head 130. Each leg 110 has a piercing end 115 and a mounting end 120. The piercing end 115 is located remote from the mounting head 130 and is adapted to pierce through body tissues and surgical implants. The mounting end 120 of each leg 110 is integrally mounted to the mounting head 130 (to be described in detail below). In some instances, the legs 110 are integrally formed with the mounting head 130. Each leg 110 is made from a memory-shaped alloy such as nickel-titanium alloy such that it is adapted (with thermal-mechanical properties) to move relative to the mounting head 130 between a piercing configuration and an anchoring configuration depending on the temperature of the alloy. The legs 110 assume a piercing position at a first temperature in which the legs 110 are configured to pierce the implant and tissues for installation of the anchor. The legs 110 assume an anchoring configuration at a higher temperature in which the legs are displaced for securing the implant and tissues in use.

The mounting head 130 of the leg 110 preferably includes an eye adapted to be engageable by a coupling device (for example a hook) of a tool for deploying and retrieving the anchor 100 in use. It is to be appreciated that the mounting head 130 can be of any suitable shape having a central aperture or eye for engaging with the coupling device, with non-limiting examples including: circular, oval and hexagonal shapes. In some embodiments, the shape of the central aperture of the head is configured to be complementary to the coupling device and it is envisaged that instead of the head 130 including an eye, it could be of other form with the coupling device of the tool being configured for co-operation with the head to achieve the desired function. It is to be appreciated that the mounting head 130 and/or the joint between the head 130 and the legs 110 is configured to have a suitable thickness or width relative to the piercing end 115 of the legs 110, such that the mounting head 130 and/or the joint between the head 130 and the legs 110 abuts the surgical implant or body tissues in use, and is thus prevented from moving through a puncture in the implant or tissue created by the installation of the surgical anchor.

The pair of legs 110 is mounted to the mounting head 130 at the mounting ends 120 such that they are located substantially parallel to each other and effectively forming a two-legged fork when the legs 110 are in the piercing configuration. In particular, the piercing ends 115 of the pair of legs 110 are proximate each other in the piercing configuration. Preferably, the distance between the pair of legs 110, including the piercing ends 115 of the legs 110, is equal to or less than 3 mm in the piercing configuration. More preferably, the distance between the piercing ends 115 of the legs 110 is equal to or less than 2 mm in the piercing configuration.

In the anchoring configuration, the pair of legs 110 is configured to bend along their lengths so that the legs 110 would trap or anchor implant and body tissues against the mounting head 130. In one embodiment, the piercing ends 115 of the pair of legs 110 extend away from each other in the anchoring configuration. In particular, the piercing ends 115 may extend away from each other in different or opposing directions so that the legs are anchored behind the surgical implant or body tissues in use. Preferably, the distance between the piercing ends 115 in the anchoring configuration is equal to or greater than 8 mm. More preferably, the distance between the piercing ends 115 in the anchoring configuration is equal to or greater than 10 mm. It is to be appreciated that the above described dimensions for the piercing and anchoring configurations are ideal dimensions for the anchor to be deployed endoscopically (for example, via an endoscopic delivery device) and for fastening an endoscopic implant to body tissues. However, other dimensions can also be used. In some embodiments, the pair of legs may be configured with different longitudinal lengths.

In a further embodiment, at least one leg 110 of the pair of legs 110 is curved along its longitudinal length to improve the ease of deployment and retrieval of the anchor in use. In some instances, the curved leg 110 is configured to curve away from the opposing leg 110 of the pair of legs 110. In other instances, both legs 110 are configured to curve away from each other.

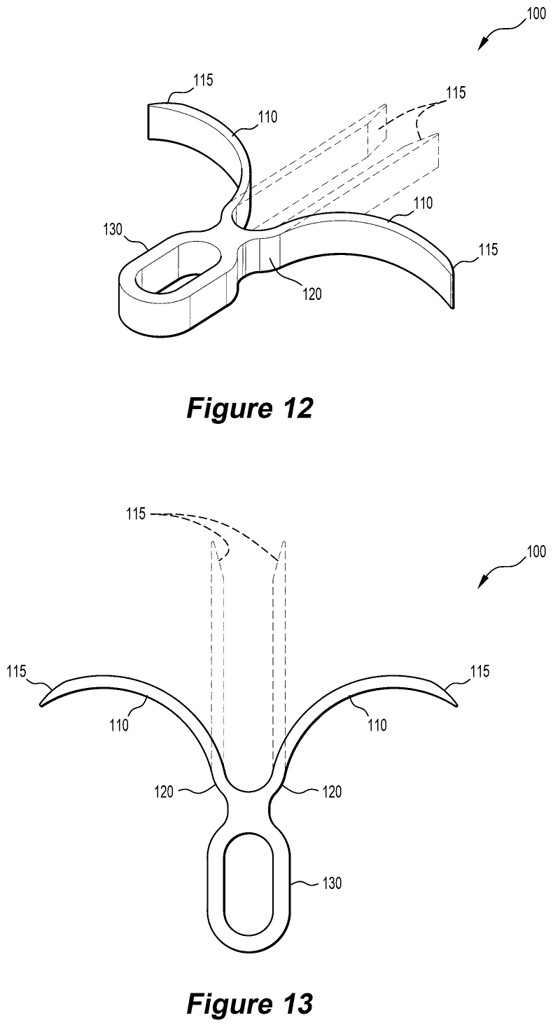

As seen in FIGS. 12 and 13, the legs may be configured to be substantially straight in the piercing configuration and substantially curved along its lengths in the anchoring configuration. In other configurations, both legs 110 are curved when in the piercing configuration. In some instances, the curvature of the leg 110 is more pronounced when the leg is in the anchoring configuration.

Referring to FIGS. 10 and 11, in yet another embodiment, at least one of the legs 110 is configured to have a kinked formation along its longitudinal length. The kink formation may be located approximately medial along the longitudinal length of the legs 110 with the legs 110 having different degrees and direction of curvature on either side of the kink. For example, a portion of the leg 110 that is on a first side of the kink (proximal to the mounting end 120) can be curved in a first direction, with a remaining portion of the leg on the other side of the kink (being proximal to the piercing end 115) curved in a second direction. In one embodiment, the two directions are opposing directions. The curved portion of the leg 110 may be configured to abut an opposing leg 110 such that the piercing ends of the pair of legs 110 are prevented from touching in the piercing configuration. This configuration allows easier deployment of the anchors by surgeons in use, where the distance between the piercing ends 115 in the piercing configuration is more consistent and therefore predictable. It is to be appreciated that in a further embodiment, the legs 110 may have two or more kinks and curved along their longitudinal lengths in three or more directions. In some instances the piercing ends 115 of a pair of legs 110 are directed toward each other in the piercing configuration to assist with the installation of the anchor in use.

The anchor 100 and its legs 110 are configured to move or transition between the piercing configuration and the anchoring configuration depending on the temperature. The legs 110 are manufactured with nickel-titanium alloys such as Nitinol which exhibits the desired thermal-mechanical memory-shape properties. The legs are manufactured with at least two stable states; a piercing configuration at a first temperature and an anchoring configuration at a second temperature. The transition of the leg 110 between the two configurations is automatic and driven by a thermal-mechanical property of the alloys during a temperature change. In one configuration, the legs 110 are in the piercing configuration when the temperature is substantially of refrigeration temperature (for example between 3 to 5 degrees Celsius) and in the anchoring configuration when the temperature is substantially of body temperature (for example, approximately 37 degrees Celsius in the case of humans). In one configuration, the piercing configuration temperature range is between 37 and 42 degrees Celsius. It is to be appreciated that the temperature range for which the anchor 100 assumes the piercing configuration can be configured to be between 0 to 8 degrees Celsius, or any other suitable temperature range that is substantially cooler than the body temperature. Likewise, the temperature range for which the anchor 100 assumes the anchoring configuration can be configured to be above room temperature, or any other suitable temperature range that is higher than the piercing temperature range so that the legs 110 will be displaced in the anchoring configuration upon installation inside a living body. Other suitable shape memory alloys that could be used instead of nickel-titanium alloys include, but are not limited to: beta titanium alloys (Ti—Nb, Ti—Mo, T-V), beta brass alloys (Cu—Zn—Al and variations thereof), bronze-based alloys (Cu—Al—Ni, Cu—Al—Be), iron-based alloys (Fe—Mn—Si, Fe—Co—Cr) and nickel-aluminium alloys.

The synergistic combination of the anchor 100 configurations and temperature-based shape-memory positions allow the anchor 100 to be suitably deployed for affixing surgical implants and body tissues during non-invasive surgical operations where a surgeon would not have sufficient manoeuvring room to adequately position and deploy conventional surgical staples. In particular, anchors 100 embodying the present invention can be used for endoscopic procedures where a surgeon would have limited manoeuvring options for fastening implants and body tissues using suitable delivery devices inside an endoscopic tube.

In use, the anchor 100 is first cooled to a first temperature such that the legs 110 are in the piercing configuration before deployment. The anchor may be cooled by submersion in suitably cold sterilised water or placed in contact with ice. The mounting head 130 of the anchor 100 may be coupled to, or grasped, by an appropriate device, for example a hook or surgical forceps, and loaded into a suitable delivery tool. The anchor 100 and the delivery tool are inserted into the appropriate body part of a patient by a surgeon endoscopically (for example, through an endoscope, gastroscope or any suitable endoscopic tube). When an appropriate deployment site is located by the surgeon, the anchor 100 is rapidly installed in its piercing configuration to the deployment site for holding implant and body tissues together. In one arrangement, a coupling device is engaged with the mounting head 130 of the anchor 100 for pushing the anchor forward into the implant and/or body tissue at the deployment site. As the anchor 100 pierces through the implant and the body tissues, the temperature of the anchor 100 reaches a higher temperature (for example normal body temperature of 37 degrees Celsius) such that the legs 110 transition to the anchoring configuration, which then trap or anchor the anchor 100 in place.

The anchor 100 in the anchoring configuration can be retrieved endoscopically using an appropriate retrieving tool. The tool may have a coupling device which is suitably cooled to the first temperature (or the piercing configuration temperature) of the anchor 100. The coupling device may be cooled by submersion in suitably cold sterilised water or placed in contact with ice. The coupling device is then inserted into the body endoscopically (for example, through an endoscope, gastroscope or any suitable endoscopic tube). The mounting head 130 of the anchor 100 is subsequently engaged or coupled with the cooled coupling device, which has the effect of cooling the temperature of the legs 110 by way of conduction. A temperature reduction of the legs 110 towards the lower temperature causes the legs 110 to transition from the anchoring configuration at least partly back towards the piercing (parallel) configuration in which the anchor 100 is subsequently retrieved by pulling the mounting head 130.

It is to be noted that the anchor 100 is not only suitable for use with the endoscopic barrier liner previously described, it is equally suitable for fastening or affixing other surgical implants to the stomach tissues such as the pylorus or duodenum regions.

A tool 200 for delivering and placing the anchor comprises a handle 205 carrying a filament 211 of sufficient length to enable it to be introduced gastroscopically into the area of the duodenum. The filament 211, although flexible, has sufficient rigidity to perform the functions to be described. The filament 211 consists of an outer sheath 219 and a core 217 which can be displaced longitudinally within the sheath. At its outer or distal end, the sheath 219 carries a substantially rigid tubular housing 218; the inner or proximate end 219a of the sheath 219 being firmly attached to the handle 205. At its outer or distal end 217a, the core 217 carries a coupling device 215 engageable with the head 130 of the surgical anchor 100. In the embodiment shown, the coupling device 215 is of hook-like form configured to engage an eye in the head 130 of the anchor although it is envisaged that different head formations and corresponding coupling devices could alternatively be adopted. Longitudinal displacement of the core 217 relative to the sheath 219 enables the coupling device with surgical anchor attached thereto to be displaced from a position in which the coupling device and head of the anchor are within the tubular housing 218 with the anchor thereby held captive to the coupling device 215, and a position in which the coupling device 215 extends beyond the forward end of the tubular housing 218 whereby it can be uncoupled from the head of the anchor by simple manipulation after placement of the anchor. In the captive condition, legs of the anchor project at least partially from the forward end of the housing 218 to enable their insertion into tissue.

Displacement of the core 217 within the sheath 219 and thereby extension or retraction of the coupling device 215 relative to the tubular housing 218 is effected by a slider 207. The slider 207 is mounted to the handle 205 and engaged with a piston 203 mounted to the inner end of the core 217 and slidable within the interior of the handle 205, which as shown is of cylindrical form. In the embodiment shown, the slider 207 is in the form of a sleeve slidably mounted on the outer surface of the handle 205 and configured to be easily gripped between the thumb and forefinger of the hand of a user.

Advantageously, a lock is provided between the slider 207 and handle 205 to lock the slider 207 in a position in which the coupling device is retracted within the tubular housing 218 and thereby to prevent its accidental extension. The lock may be a simple detent-type friction lock which releases when forward force is applied to the slider 207 by the user. During forward movement of the slider 207 on the handle 205, the slider 207 may pass through a number of intermediate locking positions which can be sensed by the user during movement of the slider 207 to enable the slider 207 to be stopped in one of a number of defined intermediate positions corresponding to different degrees of extension of the anchor from the forward housing.

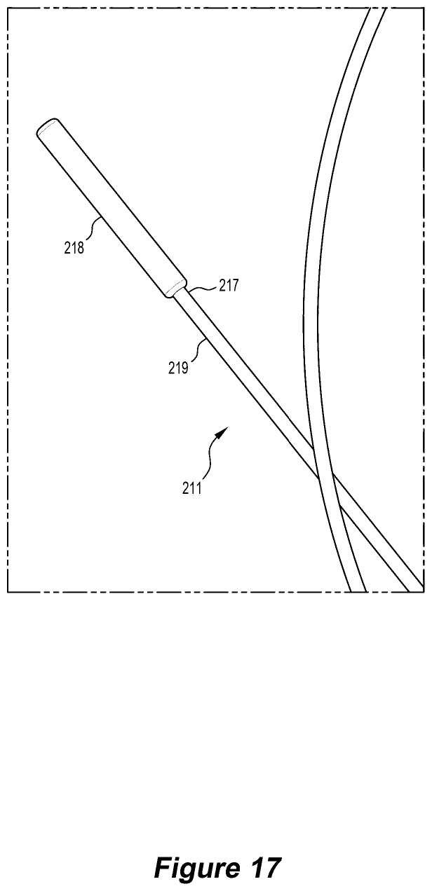

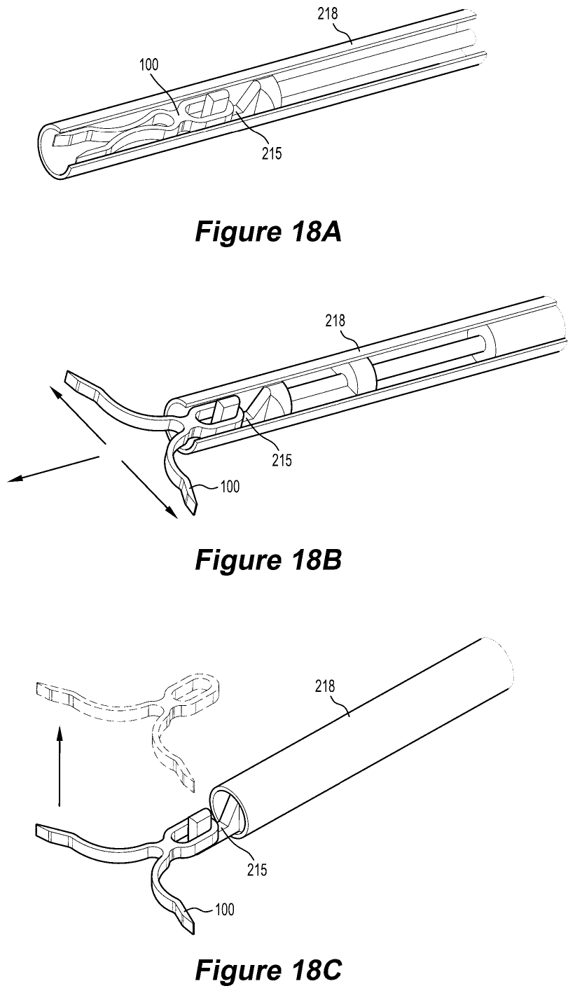

In use, the coupling device 215 is inserted through the eye of the anchor 100, the anchor having been chilled prior to use. The anchor is drawn into the housing 218 by pulling the slider 207 to the unextended position. FIG. 17 shows the condition with an anchor fully withdrawn into the housing 218. The flexible filament 211 is inserted into a patient via a gastroscope, and the distal end of the housing 218 is placed over a tissue site to which the barrier implant is to be attached. The slider 207 is brought to the extended position in order to push the coupling device 215 out of the distal end of the housing 218 to deliver an anchor to the required tissue site (FIGS. 18A to 18C). As the chilled anchor is delivered into the tissue, the temperature of the anchor equilibrates with that of its environment and deforms to its anchoring configuration.

When the anchor is required to be removed from a patient, the action shown in FIGS. 18A to 18C is reversed. First, the coupling device 215 is inserted into the eye of the anchor. Cold water is delivered down a gastro scope to the anchor to partially return the anchor to its “chilled” shape, which allows the anchor to slide out of the tissue with minimal trauma as the coupling device 215 is withdrawn into the housing 218 and removed from the patient's body.

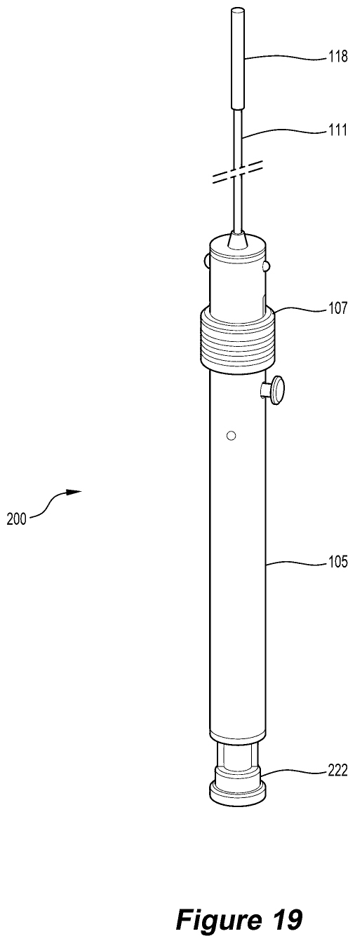

FIG. 19 illustrates an alternative embodiment of the tool in which the delivery action is spring-loaded. The tool 200 further comprises a lock 222 which can be rotated between an engaged position, in which an internal spring (not shown) is prevented from release, and a disengaged position (indicated by a groove 221), which does not inhibit the internal spring. The lock 222 is set by a user to the disengaged position 221 when loading an anchor onto the tool 200. Once the distal end of the housing 218 is placed at the required tissue site, the lock 222 is rotated to the engaged position 220 and a button 223 is pressed, which releases the internal spring (not shown) to displace the core 217 and coupling device 215 to deliver the anchor to the required tissue site.

Although the tool has been described with reference to its use in relation to the anchor 100 for fastening the barrier implant 10, it is to be understood that the tool has applicability in other surgical procedures.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A gastrointestinal barrier implant including a support, a distal barrier tube extending from the support in a first direction, and a proximal retainer extending from the support in a direction opposite to said first direction, wherein the proximal retainer is adapted to be anchored to an internal wall of the stomach of a user and to extend from the stomach through the pyloric orifice such that the support is located below the pyloric orifice, the support depending from the proximal retainer, and wherein the distal barrier tube depends from the support so as to provide a liner through the duodenum.

2. A gastrointestinal barrier implant as claimed in claim 1, wherein the support is in the form of a resilient structure.

3. A gastrointestinal barrier implant as claimed in claim 1, wherein the support is movable between a contracted condition and an expanded condition.

4. A gastrointestinal barrier implant as claimed in claim 1, wherein the support is arranged to support open the barrier implant to facilitate passage of food therethrough, when the support is in said expanded condition.

5. A gastrointestinal barrier implant as claimed in claim 1, wherein the support is in the form of a stent.

6. A gastrointestinal barrier implant as claimed in claim 1, wherein the support assists in locating the barrier implant by pushing outwardly against the duodenum.

7. A gastrointestinal barrier implant as claimed in claim 1, wherein support is arranged to prevent piercing of the wall of the duodenum.

8. A gastrointestinal barrier implant as claimed in claim 7, wherein the support is without barbs.

9. A gastrointestinal barrier implant as claimed in claim 1, wherein the proximal retainer terminates in a collar which is arranged to receive one of more anchors for anchoring the barrier implant to the stomach.

10. A gastrointestinal barrier implant as claimed in claim 1 wherein, when in use, the proximal retainer is anchored to an internal wall of the stomach of the user and extends from the stomach through the pyloric orifice to the support which is located below the pyloric orifice, the support depending from the proximal retainer, and the distal barrier tube depends from the support providing a liner through the duodenum.

11. A gastrointestinal barrier implant as in claim 1, wherein the implant is packaged into a condensed insert which is inserted into a cavity of a delivery catheter for delivery into the user, wherein the cavity retains the support in the contracted condition.

12. A gastrointestinal barrier implant as claimed in claim 11, wherein the delivery catheter is arranged to deliver the implant to the location, and includes a control to remotely eject the insert from the cavity.

13. A gastrointestinal barrier implant including a distal barrier tube and a proximal retainer wherein, when in use, the proximal retainer is anchored to an internal wall of the stomach of a user and extends from the stomach through the pyloric orifice, and the distal barrier tube depends from the proximal retainer so as to be supported in place by the proximal retainer whereby the distal barrier tube provides a liner through the duodenum.

14. A method of implanting a barrier implant including the steps of:
inserting a delivery catheter into a user;
operating the delivery catheter to remotely eject the barrier implant from the delivery catheter, with the support expanding into position below the pyloric orifice;
drawing the proximal retainer upwardly from the support, through the orifice and into the stomach; and
inserting anchors through the proximal retainer and into the stomach wall.

15. A method of implanting a barrier implant as claimed in claim 14, wherein each anchor is adapted to move between an engaging condition and a releasing condition in response to temperature.

16. A method of implanting a barrier implant as claimed in claim 15, wherein each anchor is formed from a shape memory alloy.

17. A method of implanting a barrier implant as claimed in claim 16, wherein each anchor is formed from nitinol material.

18-40. (canceled)

41. A gastrointestinal barrier implant as claimed in claim 1, wherein the support is a resilient stent.

42. A gastrointestinal barrier implant as claimed in claim 41, wherein the proximal retainer terminates in a collar which is arranged to receive one or more anchors for anchoring the barrier implant to the stomach.

43. A gastrointestinal barrier implant as claimed in claim 42, wherein the support is movable between a contracted condition and an expanded condition.
